(19) United States
(12) Patent Application Publication
(10) Pub. No.: US 2024/0261201 A1
(43) Pub. Date: Aug. 8, 2024

(54) A METHOD OF PROVIDING HIGH SPF TO A TOPICAL SURFACE OF A BODY

(71) Applicant: Conopco, Inc., d/b/a UNILEVER, Englewood Cliffs, NJ (US)

(72) Inventors: Praful Gulab Rao LAHORKAR, Bangalore (IN); Rajkumar PERUMAL, Erode District (IN); Ashish Anant VAIDYA, Bangalore (IN)

(73) Assignee: Conopco, Inc., d/b/a UNILEVER, Englewood Cliffs, NJ (US)

(21) Appl. No.: 18/560,954

(22) PCT Filed: Jun. 1, 2022
(86) PCT No.: PCT/EP2022/064908

§ 371 Date: Nov. 15, 2023

(30) Foreign Application Priority Data
Jun. 4, 2021 (EP) 21177801.4

(51) Int. Cl. A61K 8/49; A61K 8/27; A61K 8/29; A61K 8/37; A61Q 17/04; A61Q 19/10

(52) U.S. Cl. CPC A61K 8/4946; A61K 8/27; A61K 8/29; A61K 8/37; A61Q 17/04; A61Q 19/10; A61K 2800/884

(57) ABSTRACT

The present invention relates to a method of delivering high Sun Protection Factor (SPF) and UVA protection factor (UVAPF) to a topical surface of a body preferably the human body. The method more preferably provides for synergistic benefit of SPF and UVA PF over and above that which can be delivered through each of two individual steps carried out sequentially viz. washing off the skin with a specific cleansing composition followed by the step of applying a sunscreen composition on to the same skin surface. The specific cleansing composition comprises water soluble sunscreens but the leave-on composition could comprise any sunscreen.

### FIELD OF THE INVENTION

The present invention relates to a method of delivering high Sun Protection Factor (SPF) to a topical surface of a body preferably the human body. The method more preferably provides for synergistic benefit of SPF over and above that which can be deliver through each of two individual steps carried out sequentially viz. washing off the skin with a specific cleansing composition followed by the step of applying a sunscreen composition on to the same skin surface.

### BACKGROUND OF THE INVENTION

Solar radiation includes about 5% ultraviolet (UV) radiation, wavelength of which is between 200 nm and 400 nm. It is further classified into three regions: from 320 to 400 nm (UV-A), 290 to 320 nm (UV-B) and from 200 to 290 nm (UV-C). Exposure to UV-A and UV-B radiation for short period is known to cause reddening of the skin and localized irritation. Continued and prolonged exposure can lead to sunburn, melanoma and formation of wrinkles. It is also reported that UV radiation causes significant damage to hair. Therefore, people desire to protect their skin and hair from the harmful effects of both UV-A and UV-B radiation.

Various cosmetic preparations that one can apply on to the skin such as creams, lotions or gels have been reported for preventing and/or protecting the skin from harmful effects of ultraviolet radiation. These cosmetic compositions usually comprise different types of organic sunscreen agents, especially ones capable of absorbing the UV-A and/or UV-B radiation present in the sun's rays. Thus, both UV-A and UV-B sunscreens are usually incorporated in photo-protective compositions so as to provide protection over the entire range of UV radiation. Such leave-on compositions are applied on to skin before a person goes outdoors and they remain thereon till the person has a next wash.

An alternate method of application of sunscreens on the skin is through wash-off products like soaps, face wash or body wash products where the sunscreens are incorporated in the skin cleansing products and are delivered on to the skin concurrent with the washing process. However, it is an extremely difficult challenge to deliver sunscreens on to skin when the primary purpose of wash-off products is to remove the dirt and oils from the skin surface and together with that, the actives in the wash-off products are also highly likely to get washed away. Thus, enhanced deposition of actives through wash-off products is an on-going challenge.

The present inventors with their years of experience in understanding the nature of available sunscreens; the knowledge of interfacial science of surfactant systems and through extensive experimentation set out find a solution to the problem of delivering high SPF on to skin. They hit upon a unique two step method that provides for synergistic improvement in SPF as compared to each of the steps individually. The method comprises the step of washing the skin with a composition comprising water soluble sunscreens followed by the step of applying a leave-on sunscreen composition on to skin.

EP1261688 (Unilever, 2001) discloses a personal wash composition which deposit high levels of sunscreen (SPF>2) while maintaining good lather (i.e. suffer minimal lather degradation over time relative to compositions with more “oily” sunscreens). Enhanced deposition is found from both bar and liquid compositions and is based on the solubility or non-solubility of the sunscreen used.

The present inventors have found that there is a limit to the SPF that can be achieved with either a wash-off sunscreen composition or a leave-on sunscreen composition, the latter being more efficacious. Incorporation of higher amount of sunscreens as taught in the prior art does not, in general, provide for continuous enhancement in the deposition efficacy and thereby the SPF achieved.

It is therefore an object of the present invention to obviate at least some drawbacks of the prior art and provide a method which delivers enhanced and even synergistic SPF as compared to the known art.

It is another object of the present invention to provide for a method that provides SPF of higher than 15 and a UVAPF (UV-A protection factor) of higher than 4.

### SUMMARY OF THE INVENTION

According to the first aspect of the present invention, there is provided a method of providing high SPF to a topical surface of a body comprising the steps of
(a) Washing the surface with a wash-off composition comprising
(i) 0.1 to 10 wt % of a water-soluble UVA sunscreen;
(ii) 0.1 to 10 wt % of a water-soluble UVB sunscreen;
(iii) 3 to 80 wt %, preferably 6 to 80 wt % surfactant; and
(iv) a cosmetically acceptable carrier;
preferably diluted with water followed by
(b) rinsing said surface with water; followed by
(c) applying a leave-on composition comprising a sunscreen and a cosmetically acceptable vehicle, on said surface,
   wherein the solubility in water of said water-soluble UVA sunscreen and said water-soluble UVB sunscreen is higher than 10 g/l at 25° C.

The second aspect of the present invention relates to a kit comprising
(a) A wash-off composition comprising 0.1 to 10 wt % of a water soluble UVA sunscreen, 0.1 to 10 wt % of a water soluble UVB sunscreen, 3 to 80 wt %, preferably 6 to 80 wt % surfactant, and a cosmetically acceptable carrier,
(b) A leave-on composition comprising a sunscreen and a cosmetically acceptable vehicle; and
(c) Instructions for use.

### DETAILED DESCRIPTION OF THE INVENTION

These and other aspects, features and advantages will become apparent to those of ordinary skill in the art from a reading of the following detailed description and the appended claims. For the avoidance of doubt, any feature of one aspect of the present invention may be utilized in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated. Except in the operating and comparative examples, or where otherwise explicitly indicated, all numbers in this description and claims indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated. The disclosure of the invention as found herein is to be considered to cover all embodiments as found in the claims as being multiply dependent upon each other irrespective of the fact that claims may be found without multiple dependency or redundancy. Where a feature is disclosed with respect to a particular aspect of the invention (for example a method of the invention), such disclosure is also to be considered to apply to any other aspect of the invention (for example a kit of the invention) mutatis mutandis.

By “a wash-off composition” as used herein, is meant to include a composition for cleaning topical surfaces of mammals, especially humans. This composition is particularly useful for use on the sun-exposed parts of the body. Such a composition is generally of the rinse off type which means that the composition is high in surfactants which are known to help in cleaning surfaces to make them free of oils and dirt. The wash-off composition is generally used by diluting with water as it is applied on to skin, scalp or hair, after which the consumer works up a lather to ensure that the dirt and oil on the surface are solubilized in the micelles of the surfactant solution and the body is then rinsed with copious amounts of water to ensure that the surface is substantially free of the composition. The wash-off composition of the present invention can be in the form of a liquid, lotion, cream, gel, shampoo, conditioner or soap bar. “Skin” as used herein is meant to include skin on the face and body (e.g., neck, chest, back, arms, underarms, hands, legs, buttocks and scalp) and especially to the sun exposed parts thereof. The wash-off composition of the invention is also of relevance to application on any other keratinous substrates of the human body other than skin e.g. scalp and hair where products may be formulated with specific aim of providing photo-protection. It has been observed that with the use of the wash-off composition of the method of the present invention, the dirt and oil on the skin surface are substantially removed during the washing and rinsing process. Further, surprisingly specific combination of the sunscreens ensures that they deposit in much higher amounts than is generally expected from rinse off products, in such a way that the deposited sunscreens interact synergistically with the sunscreens applied from the leave-on composition to provide the enhanced SPF and UVAPF benefit. The enhanced deposition leads to delivering high Sun Protection Factor (SPF). By high SPF as per this invention is meant a composition that has an SPF at least 15, preferably higher than 20, more preferably higher than 30, further more preferably higher than 50, further more preferably higher than 80, and in optimum cases higher than 100. Most preferably the invention provides for SPF in the range of 20 to 50. The SPF could be as high as 160. The SPF in the present invention is measured using transmittance measurement technique. The method of the invention also delivers high UVAPF. By high UVAPF is meant that the method delivers UVAPF (initial) of at least 4, preferably higher, higher than 8, further preferably higher than 12, further more preferably higher than 16, and most preferably higher than 20. In especially preferred aspects, the present invention deliver UVAPF value in the range of 4 to 16. The UVAPF could be as high as 40.

The wash-off composition which is used in the first step of the method of the invention comprises a water-soluble UVA sunscreen; a water soluble UVB sunscreen; 3 to 80 wt % preferably 6 to 80 wt % surfactant; and a cosmetically acceptable carrier. By water soluble sunscreen, whether of the UVA type, or of the UVB type, is meant that the solubility in water of the sunscreen is higher than 10 g/l preferably higher than 50 g/l at 25° C. Preferably, the solubility in water of the sunscreen is as high as 200 g/l at 25° C.

The preferred UVB sunscreen for use in the wash-off composition is phenyl benzimidazole sulphonic acid (PBSA) or benzylidene camphor sulfonic acid, preferably PBSA.

PBSA has the chemical structure:

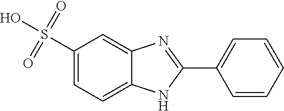 [SMILES: O=S(=O)(O)c1ccc2[nH]c(-c3ccccc3)nc2c1]

### 2-Phenylbenzimidazole 5-sulfonic acid

PBSA also known as Ensulizole is commercially available as Eusolex 232 (from Merck KGaA). PBSA is also available under the brand names Neo Heliopan Hydro (from Symrise), Parsol HS (from DSM) and Sunsafe ES (from Uniproma). It is generally used in the water solubilized form after neutralization to deliver desired benefit.

Benzylidene camphor sulfonic acid has the chemical structure: It is generally used in the water solubilized form pre/post neutralization to deliver desired benefit.

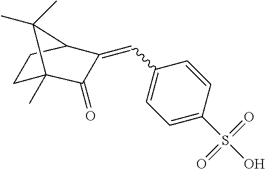 [SMILES: CC12CCC(C(=Cc3ccc(S(=O)(=O)O)cc3)C1=O)C2(C)C]

The preferred UVA sunscreen for use in the wash-off composition is one or more of disodium phenyl dibenzimidazole tetrasulfonate (Neoheliopan AP) or terephthalylidene dicamphor sulfonic acid (TDSA).

Disodium phenyl dibenzimidazole tetra sulfonate also known as bisdisulizole disodium has the chemical structure as given below:

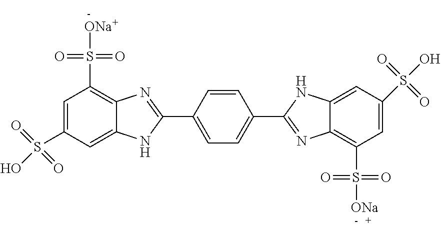

This is commercially available as Neo heliopan AP (from Symrise Shanghai Ltd) or as Sunsafe DPDT (from Uniproma), It is generally used in the water solubilized form after neutralization to deliver desired benefit.

Terephthalylidene dicamphor sulfonic acid (TDSA) has the structure

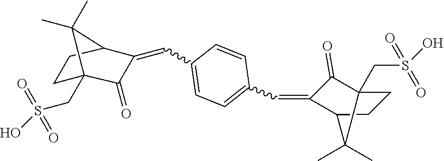 [SMILES: CC1(C)C2CCC1(CS(=O)(=O)O)C(=O)C2=Cc1ccc(C=C2C(=O)C3(CS(=O)(=O)O)CCC2C3(C)C)cc1]

It is also known as Ecamsule. This is commercially available as Mexoryl SX (U.S. Pat. No. 4,585,597) by L' Oreal or Sunsafe TDSA (from Uniproma). It can be used as parent acid or its salts in water solubilized form to deliver the desired benefit.

The wash-off composition preferably comprises 0.1 to 10 wt %, more preferably 0.25 to 8 wt %, even more preferably 0.5 to 5% water soluble UVA sunscreen. The composition preferably comprises 0.1 to 10 wt %, more preferably 0.25 to 8 wt %, even more preferably 0.5 to 5% water soluble UVB sunscreen. There are certain sunscreens which have both a UVA screening efficacy as well as a UVB screening efficacy. Such sunscreens provide both UVA and UVB protection e.g. benzophenone-4. Benzophenone-4 (Sulisobenzone) has the chemical structure:

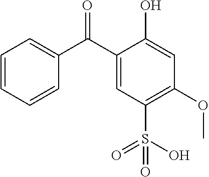 [SMILES: COc1cc(O)c(C(=O)c2ccccc2)cc1S(=O)(=O)O]

Such sunscreens may also be included in the wash-off composition of the invention. When included, it is to be understood that it is taken as having included one type of sunscreen i.e. either a UVA sunscreen or a UVB sunscreen and not both. Thus, when included as a UVA sunscreen, it is essential to include another sunscreen as a UVB sunscreen. Similarly, when included as a UVB sunscreen, it is essential to include another sunscreen as a UVA sunscreen. Preferably, benzophenone-4 is used as a UVB water soluble sunscreen in the wash-off composition.

The water-soluble sunscreens for inclusion in the wash-off composition of the present invention are generally commercially available in the acid form. When included in the composition in the acid form (pre/partially neutralized forms) i.e having sulphonic acid group (—SO₃H) the was-off composition additionally comprises a neutralising agent to convert the acid form in to the salt form, in which form it is known to be active as a sunscreen with an exception to Mexoryl SX which can act as sunscreen with and without neutralization. When included, the neutralising agent is preferably in 0.05% to 4% by weight of the composition. The neutralising agent is preferably an inorganic or an organic alkali. Organic alkali is preferably an amine such as triethanol amine or diethanol amine. The present inventors have observed that the inorganic alkali is especially preferred. Preferred are alkali metal hydroxides. Most preferred metal hydroxide for inclusion as neutralising agent in the composition of the invention are sodium hydroxide or potassium hydroxide.

One of the objects of the first step of the method of the invention is to ensure that the sunscreen is deposited in as high an amount as possible on the skin through the wash-off process. Without wishing to be bound by theory, the inventors believe that this is achieved through the select combination of sunscreens chosen by their structure and solubility which determine their partition between rinse away water and cleansing surface resulting in high sunscreen deposition.

The wash-off composition of the invention comprises a surfactant which aids in the cleaning action. To enable cleaning, surfactant is included in 3 to 80% preferably 6 to 80%, further more preferably 10 to 75% by weight of the composition.

### Skin Cleansing Compositions

The surfactant is preferably an anionic surfactant e.g. an alkyl sulphate and/or ethoxylated alkyl sulfate surfactant. These anionic surfactants are preferably present at a level of from 1 to 20%, preferably 2 to 16%, more preferably from 3 to 16%, further more preferably 4 to 15%, even more preferably 5 to 14%, still more preferably 6 to 12% and still more preferably 8 to 10% by weight of the wash-off composition. Preferred alkyl sulfates are C8-18 alky sulfates, more preferably C12-18 alkyl sulfates, preferably in the form of a salt with a solubilising cation such as sodium, potassium, ammonium or substituted ammonium. Preferred alkyl ether sulfates are those having the formula: RO(CH₂CH₂O)ₙSO₃M; wherein R is an alkyl or alkenyl having from 8 to 18 (preferably 12 to 18) carbon atoms; n is a number having an average value of greater than at least 0.5, preferably between 1 and 3, more preferably between 2 and 3; and M is a solubilising cation such as sodium, potassium, ammonium or substituted ammonium. An example is sodium lauryl ether sulfate (SLES). SLES having an average degree of ethoxylation of from 0.5 to 3, preferably 1 to 3 is especially preferred. The wash-off composition may comprise one or more further anionic cleansing surfactants which are cosmetically acceptable and suitable for topical application.

The wash-off composition of the invention preferably additionally comprises an amphoteric surfactant preferably a betaine surfactant preferably an alkyl amidopropyl betaine surfactant for example cocamidopropyl betaine CAPB). In a preferred embodiment, the wash-off composition comprises from 0.1 to 10 wt. %, preferably from 0.5 to 8 wt. %, more preferably from 1 to 5 wt. % of a betaine surfactant

SLES and CAPB are often combined for use in shampoo and bodywash formulations. Alternately the wash-off composition may be formulated as a soap bar or liquid soap bodywash.

Soap is a suitable surfactant for personal washing applications of wash-off composition used in the present invention. The soap is preferably C8-C24 soap, more preferably C10-C20 soap and most preferably C12-C16 soap. The soap may or may not have one or more carbon-carbon double bond or triple bond. The cation of the soap may be alkali metal, alkaline earth metal or ammonium. Preferably, the cation of the soap is selected from sodium, potassium or ammonium. More preferably the cation of the soap is sodium or potassium.

The soap may be obtained by saponifying a fat and/or a fatty acid. The fats or oils generally used in soap manufacture may be such as tallow, tallow stearines, palm oil, palm stearines, soya bean oil, fish oil, castor oil, rice bran oil, sunflower oil, coconut oil, babassu oil, palm kernel oil, and others. In the above process the fatty acids are derived from oils/fats selected from coconut, rice bran, groundnut, tallow, palm, palm kernel, cotton seed, soyabean, castor etc.

A typical fatty acid blend consisted of 5 to 30% coconut fatty acids and 70 to 95% fatty acids ex hardened rice bran oil. Fatty acids derived from other suitable oils/fats such as groundnut, soybean, tallow, palm, palm kernel, etc. may also be used in other desired proportions. The most preferred soap is a laurate soap. The soap, when present in solid forms of wash-off composition is present in an amount of 30 to 90%, preferably from 50 to 85%, more preferably 55 to 75% by weight of the wash-off composition. The soap, when present in liquid/semi liquid forms of the wash-off composition is present in 0.1% to 25%, preferably 0.5 to 20%, more preferably from 1 to 10% by weight of the wash-off composition.

Alternatively the surfactants are non-ionic surfactants, such as C8-C22, preferably C8-C16 fatty alcohol ethoxylates, comprising between 1 and 8 ethylene oxide. Alkyl polyglucoside may also be present in the composition, preferably those having a carbon chain length between C6 and C16. Suitable nonionic surfactant concentrations in liquid forms of cleaning application are generally more than 0.5 but less than 30%, preferably from 1 to 20%, more preferably 4 to 18%, even more preferably 5 to 15%, still more preferably 6 to 12% and still more preferably 8 to 10% by weight of the wash-off composition. In solid compositions, the surfactant is preferably present in 30 to 80%, preferably from 50 to 80% by weight of the wash-off composition.

The wash-off composition of the invention comprises a cosmetically acceptable carrier. The cosmetically acceptable carrier is preferably an aqueous solution/dispersion of cosmetically acceptable adjuvants. Water is generally included in the wash-off composition of the invention.

When the wash-off composition is in solid form e.g. in the form of a soap bar, the amount of water is in the range 14 to 25%, preferably 15 to 22% by weight of the composition. When the wash-off composition is formulated in liquid, emulsion or gel form, the water content is generally in the range of 50 to 95%, preferably in the range of 60 to 85% by weight of the composition.

Additional adjuvants which may be used to make up the cosmetically acceptable carrier of the wash-off composition are described below.

When made as a soap bar composition it may optionally comprise 2 to 15%, preferably 4 to 12% by weight of free fatty acids. By free fatty acids is meant a carboxylic acid comprising a hydrocarbon chain and a terminal carboxyl group bonded to an H. Suitable fatty acids are C8 to C22 fatty acids. Preferred fatty acids are C12 to C18, preferably predominantly saturated, straight-chain fatty acids. However, some unsaturated fatty acids or hydroxylated saturated fatty acid such as 12 Hydroxy stearic acid can also be employed.

In the form of a soap bar, the composition generally comprises electrolyte. Electrolytes as per this invention include compounds that substantially dissociate into ions in water. Suitable electrolytes for inclusion in the soap making process are alkali metal salts, or mono or di or tri salts of alkaline earth metals or other metals. Preferred alkali metal salts include sodium sulfate, sodium chloride, sodium acetate, sodium citrate, potassium chloride, potassium sulfate, sodium carbonate, more preferred electrolytes are sodium chloride, sodium sulfate, sodium citrate, potassium chloride and especially preferred electrolyte is sodium chloride, sodium sulphate, sodium citrate or a combination thereof. Electrolyte is preferably included in 0.1 to 6%, more preferably 0.5 to 6%, even more preferably 0.5 to 5%, further more preferably 0.5 to 3%, and most preferably 1 to 3% by weight of the wash-off composition. The same electrolyte range is also applicable to liquid cleansers.

The wash-off composition preferably comprises a polyhydric alcohol (also called polyol) or mixture of polyols. Polyol is a term used herein to designate a compound having multiple hydroxyl groups (at least two, preferably at least three) which is highly water soluble. Many types of polyols are available including: relatively low molecular weight short chain polyhydroxy compounds such as glycerol and propylene glycol; sugars such as sorbitol, manitol, sucrose and glucose; modified carbohydrates such as hydrolyzed starch, dextrin and maltodextrin, and polymeric synthetic polyols such as polyalkylene glycols, for example polyoxyethylene glycol (PEG) and polyoxypropylene glycol (PPG). Especially preferred polyols are glycerol, sorbitol and their mixtures. Most preferred polyol is glycerol. Preferably, the wash-off composition e.g. a face wash composition, comprises 1 to 25% by wt., more preferably 3 to 22% by wt. even more preferably 5 to 20% by wt., further more preferably from 8 to 15% by wt. and still more preferably from 10 to 13% by wt. polyol. In a preferred embodiment, the bars of the invention comprise 0 to 8%, preferably 1 to 7.5% by wt. polyol.

Suitable starchy materials which may be used include natural starch (from corn, wheat, rice, potato, tapioca and the like), pregelatinized starch, various physically and chemically modified starch and mixtures thereof. By the term natural starch is meant starch which has not been subjected to chemical or physical modification—also known as raw or native starch. The raw starch can be used directly or modified during the process of making the bar composition such that the starch becomes gelatinized, either partially or fully gelatinized.

The adjuvant system may optionally include insoluble particles comprising one or a combination of materials. By insoluble particles is meant materials that are present in solid particulate form and suitable for personal washing. Preferably, there are mineral (e.g., inorganic) or organic particles.

The insoluble particles should not be perceived as scratchy or granular and thus should have a particle size less than 300 microns, more preferably less than 100 microns and most preferably less than 50 microns.

Preferred inorganic particulate material includes talc and calcium carbonate. Talc is a magnesium silicate mineral material, with a sheet silicate structure and a composition of Mg₃Si₄(OH)₂₂, and may be available in the hydrated form. It has a plate-like morphology, and is essentially oleophilic/hydrophobic, i.e., it is wetted by oil rather than water.

Calcium carbonate or chalk exists in three crystal forms: calcite, aragonite and vaterite. The natural morphology of calcite is rhombohedral or cuboidal, acicular or dendritic for aragonite and spheroidal for vaterite.

Examples of other optional insoluble inorganic particulate materials include aluminates, silicates, phosphates, insoluble sulfates, borates and clays (e.g., kaolin, china clay) and their combinations.

Organic particulate materials include: insoluble polysaccharides such as highly crosslinked or insolubilized starch (e.g., by reaction with a hydrophobe such as octyl succinate) and cellulose; synthetic polymers such as various polymer lattices and suspension polymers; insoluble soaps and mixtures thereof.

It is preferred that the wash-off composition of the invention comprise polymers. Polymers of the acrylate class are especially preferred. Preferred bars include 0.05 to 5% acrylates. More preferred bars include 0.01 to 3% acrylates. Examples of acrylate polymers include polymers and copolymers of acrylic acid crosslinked with polyallylsucrose as described in U.S. Pat. No. 2,798,053 which is herein incorporated by reference. Other examples include polyacrylates, acrylate copolymers or alkali swellable emulsion acrylate copolymers, hydrophobically modified alkali swellable copolymers, and crosslinked homopolymers of acrylic acid. Examples of such commercially available polymers are: ACULYN®, CARBOPOL®, and CARBOPOL® Ultrez grade series.

Bar forms of the wash-off compositions preferably comprise 0.1 to 25% by wt. of bar composition, preferably 5 to 15 by wt. of these mineral or organic particles.

An opacifier may be optionally present in the wash-off composition. When opacifiers are present, the cleansing bar is generally opaque. Examples of opacifiers include titanium dioxide, zinc oxide and the like. A particularly preferred opacifier that can be employed when an opaque soap composition is desired is ethylene glycol mono- or di-stearate, for example in the form of a 20% solution in sodium lauryl ether sulphate. An alternative opacifying agent is zinc stearate.

The product can take the form of a water-clear, i.e. transparent soap, in which case it will not contain an opacifier.

The pH of preferred soaps bars of the invention is from 7 to 11, more preferably 9 to 11. Products in the liquid form which generally comprises synthetic surfactants preferably have a pH of 5.5 to 8, more preferably 6 to 8.

A preferred wash-off composition may additionally include up to 30 wt % benefit agents. Preferred benefit agents include moisturizers, emollients, sunscreens and anti-ageing compounds. The agents may be added at an appropriate step during the process of making the bars. Some benefit agents may be introduced as macro domains.

Other optional ingredients like anti-oxidants, perfumes, polymers, chelating agents, colourants, deodorants, dyes, enzymes, foam boosters, germicides, anti-microbials, lathering agents, pearlescers, skin conditioners, stabilizers or superfatting agents, may be added in suitable amounts in the process of the invention. Preferably, the ingredients are added after the saponification step. Sodium metabisulphite, ethylene diamine tetra acetic acid (EDTA), borax or ethylene hydroxy diphosphonic acid (EHDP) are preferably added to the formulation.

The wash-off composition of the invention could be used to deliver antimicrobial benefits. Antimicrobial agents that are preferably included to deliver this benefits include oligodynamic metals or compounds thereof. Preferred metals are silver, copper, zinc, gold or aluminium. Silver is particularly preferred. In the ionic form it may exist as a salt or any compound in any applicable oxidation state. Preferred silver compounds are silver oxide, silver nitrate, silver acetate, silver sulfate, silver benzoate, silver salicylate, silver carbonate, silver citrate or silver phosphate, with silver oxide, silver sulfate and silver citrate being of particular interest in one or more embodiments. In at least one preferred embodiment the silver compound is silver oxide. Oligodynamic metal or a compound thereof is preferably included in 0.0001 to 2%, preferably 0.001 to 1% by weight of the wash-off composition. Alternately an essential oil antimicrobial active may be included in the composition of the invention. Preferred essential oil actives which may be included are terpineol, thymol, carvacol, (E)-2(prop-1-enyl) phenol, 2-propylphenol, 4-pentylphenol, 4-sec-butylphenol, 2-benzyl phenol, eugenol or combinations thereof. Further more preferred essential oil actives are terpineol, thymol, carvacrol or thymol, most preferred being terpineol or thymol and ideally a combination of the two. Essential oil actives are preferably included in 0.001 to 1%, preferably 0.01 to 0.5% by weight of the wash-off composition.

### Hair Care Compositions: Shampoos and Conditioners:

As per another aspect of the invention, the wash-off composition may be used for hair care. One medium for hair cleansing and care is a shampoo. The shampoo compositions of the invention are generally formulated with an anionic surfactant e.g. an alkyl sulphate and/or ethoxylated alkyl sulfate surfactant. These anionic surfactants are preferably present at a level of from 1 to 20%, preferably 2 to 16%, further more preferably from 3 to 16% by weight of the hair care composition. Preferred alkyl sulfates are C8-18 alky sulfates, more preferably C12-18 alkyl sulfates, preferably in the form of a salt with a solubilising cation such as sodium, potassium, ammonium or substituted ammonium.

Preferred alkyl ether sulfates are those having the formula: RO(CH₂CH₂O)ₙSO₃M; wherein R is an alkyl or alkenyl having from 8 to 18 (preferably 12 to 18) carbon atoms; n is a number having an average value of greater than at least 0.5, preferably between 1 and 3, more preferably between 2 and 3; and M is a solubilising cation such as sodium, potassium, ammonium or substituted ammonium. An example is sodium lauryl ether sulfate (SLES). Preferred ethoxylated alkyl sulfate anionic surfactant is sodium lauryl ether sulfate (SLES). SLES having an average degree of ethoxylation of from 0.5 to 3, preferably 1 to 3 is especially preferred.

Shampoo compositions according to the invention may comprise one or more further anionic cleansing surfactants which are cosmetically acceptable and suitable for topical application to the hair.

A composition of the invention preferably additionally comprises an amphoteric surfactant preferably a betaine surfactant preferably an alkyl amidopropyl betaine surfactant for example cocamidopropyl betaine. In a preferred embodiment, the composition comprises from 0.1 to 10 wt. %, preferably from 0.5 to 8 wt. %, more preferably from 1 to 5 wt. % of a betaine surfactant.

To enhance deposition of actives from compositions of the invention especially shampoos, cationic polymers are generally included therein. In the present invention too, it is preferred that the composition additionally includes 0.01 to 2.0% of a cationic polymer. The cationic polymer is preferably guar hydroxypropyl trimonium chloride. Guar polymer predominantly contains galactomannan polymer chains. This polymer is available at various molecular weights and degree of cationic substitutions depending on how much the guar has been hydrolysed and cationised. The cationic polymer is preferably present in 0.04 to 0.5%, more preferably 0.08 to 0.25% by weight of the composition.

When conditioning benefits are also to be delivered through the wash-off composition it is called a hair conditioner. Typically, the most popular conditioning agents used in hair care compositions are water-insoluble oily materials such as mineral oils, naturally occurring oils such as triglycerides and silicone polymers. Conditioning benefit is achieved by the oily material being deposited onto the hair resulting in the formation of a film, which makes the hair easier to comb when wet and more manageable when dry. An especially useful conditioning agent is a silicone compound, preferably a non-volatile silicone compound. Advantageously compositions herein may include one or more silicones. The silicones are conditioning agents found in dispersed or suspended particulate form. They are intended to deposit onto hair remaining behind after rinsing of the hair with water. Suitable silicone oils may include polyalkyl siloxanes, polyaryl siloxanes, polyalkylaryl siloxanes, polyether siloxane copolymers and mixtures thereof. Amino silicones are often formulated with shampoo compositions. Amino silicones are silicones containing at least one primary amine, secondary amine, tertiary amine or a quaternary ammonium group. High molecular weight silicone gums can also be utilized. Another useful type are the crosslinked silicone elastomers such as Dimethicone/Vinyl/Dimethicone Crosspolymers (e.g. Dow Corning 9040 and 9041).

Amounts of the silicone in compositions where present may range from about 0.1 to about 10 wt. %, preferably from about 0.1 to about 8 wt. %, more preferably from about 0.3 to about 5 wt. % by weight of the hair care compositions.

The pH of the composition is preferably equal to or higher than 4.0, more preferably in the range of 5.0 to 7.0.

The hair conditioning composition usually comprises conditioning surfactants selected from cationic surfactants, used singly or in admixture. Suitable cationic surfactants for use in conditioner compositions according to the invention include cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, dihydrogenated tallow dimethyl ammonium chloride (eg, Arquad 2HT/75 from Akzo Nobel), cocotrimethylammonium chloride, PEG-2-oleammonium chloride and the corresponding hydroxides thereof. Further suitable cationic surfactants include those materials having the CTFA designations Quaternium-5, Quaternium-31 and Quaternium-18. Mixtures of any of the foregoing materials may also be suitable. A particularly useful cationic surfactant for use in conditioners is cetyltrimethylammonium chloride, available commercially, for example as GENAMIN CTAC, ex Hoechst Celanese. Another particularly useful cationic surfactant for use in conditioners is behenyltrimethylammonium chloride, available commercially, for example as GENAMIN KDMP, ex Clariant. Yet another preferred cationic surfactant is stearamidopropyl dimethylamine.

The most preferred cationic surfactants for use in the composition are stearamidopropyl dimethylamine, behentrimonium chloride, or stearyl trimethyl ammonium chloride. In conditioners of the invention, the level of cationic surfactant will generally range from 0.1% to 5%, preferably 0.5 to 2.5% by weight of the hair care composition.

Hair conditioning compositions preferably may also additionally comprise a fatty alcohol. The combined use of fatty alcohols and cationic surfactants in conditioning compositions is believed to be especially advantageous, because this leads to the formation of a lamellar phase, in which the cationic surfactant is dispersed.

Hair care compositions whether delivered as shampoos or conditioners usually comprise an anti-dandruff agent. The most preferred anti-dandruff agent for use in the composition is a zinc based anti-dandruff agent preferably zinc pyrithione. Shampoo composition as per the invention preferably additionally or alternately comprises a conazole fungicide. Preferably the conazole fungicide is selected form ketoconazole, climbazole or mixtures thereof. The azole fungicide is preferably included in 0.01 to 2%, more preferably 0.025 to 0.75% by weight of the composition. The presence of a conazole fungicide is believed to improve the deposition of zinc pyrithione.

The present invention includes a second step of the method which comprises applying a leave-on composition comprising a sunscreen and a cosmetically acceptable vehicle on to the desired topical surface e.g a skin surface. By leave-on composition is meant a composition that is applied on the desired topical surface and left thereon till such time that the surface is cleaned usually after several hours or after one day when a person normally has his bath or shower or any other personal cleansing routine. The leave-on composition of the present invention can be in the form of a liquid, lotion, cream, spray, stick or gel. “Skin” as used herein is meant to include skin on the face and body (e.g., neck, chest, back, arms, underarms, hands, legs, buttocks and scalp) and especially to the sun exposed parts thereof. The leave-on composition of the invention is also of relevance to application on any other keratinous substrates of the human body other than skin e.g. scalp and hair where products may be formulated with specific aim of providing photo-protection.

The sunscreen in the leave-on composition is an organic UVA sunscreen, an organic UVB sunscreen, an inorganic sunblock or mixtures thereof preferably a combination of an organic UVB sunscreen and an organic UVA sunscreen.

Preferably, the leave-on composition comprises a UVA organic sunscreen that absorbs UVA radiations and prevent them from reaching a surface e.g. skin of a user. Examples of sunscreens that may be used as UVA organic sunscreen in the composition are dibenzoylmethane compound, bisdisulizole disodium (commercially available as Neo Heliopan® AP), diethylamino hydroxybenzoyl hexyl benzoate (commercially available as Uvinul® A Plus), Ecamsule (commercially available as Mexoryl SX), Dimethoxyphenyl-[1-(3,4)]-4,4-dimethyl 1,3-pentanedione, and Methyl anthranilate. Preferably, sunscreen that may be used as UVA organic sunscreen in the leave-on composition is selected from a dibenzoylmethane compound or diethylamino hydroxybenzoyl hexyl benzoate, preferably a dibenzoylmethane compound. Examples of sunscreen of dibenzoymethane compound that may be used as UVA organic sunscreen in the leave-on composition include butyl methoxydibenzoylmethane or BMDM (commercially available as Parsol® 1789 or Avobenzone), 2-methyldibenzoylmethane, 4-isopropyldibenzoyl-methane, 4-tert-butyldibenzoylmethane, 2,4-dimethyldibenzoylmethane, 2,5-dimethyldibenzoylmethane, 4,4′-diisopropyl-dibenzoylmethane, 2-methyl-5-isopropyl-4′-methoxydibenzoylmethane, 2-methyl-5-tert-butyl-4′-methoxy-dibenzoyl methane, 2,4-dimethyl-4′-methoxy dibenzoylmethane or 2,6-dimethyl-4-tert-butyl-4′-methoxy-dibenzoylmethane. Most preferably, dibenzoylmethane compound that may be used as UVA organic sunscreen is BMDM.

Preferably, the leave-on composition comprises from 0.01 to 10 wt %, more preferably from 0.1 to 8 wt %, even more preferably from 1 to 6 wt %, further more preferably 1 to 5 wt %, still more preferably from 1 to 3 wt %, yet more preferably from 1 to 2.5 wt % of one or more UVA organic sunscreens.

Preferably, the leave-on composition may comprise a UV-B organic sunscreen. UVB organic sunscreens absorb UVB radiations and prevent them from reaching a surface e.g. skin of a user. Examples of UVB organic sunscreens that may be used in the composition include compounds from the class of cinnamic acid, salicylic acid, diphenyl acrylic acid, Benzylidene camphor and derivatives thereof. Examples of such compounds include 2-ethylhexyl salicylate (commercially available as Octisalate™), 3,3,5-Trimethylcyclohexyl 2-hydroxybenzoate (commercially available as Homosalate™), Ethylhexyl Methoxycinnamate (commercially available as NeoHelipan® AV), 2-ethylhexyl 2-cyano-3,3-diphenylacrylate (OCR; commercially available as Octocrylene™), 2-Hydroxy-4-methoxybenzophenone (commercially available as Oxybenzone™), 2-ethyl-hexyl-4-methoxy cinnamate (MCX; commercially available as Parsol MCX™), 3-Benzylidene camphor, Benzylidene camphor sulfonic acid, DEA methoxycinnamate, Cinoxate, Diethylhexyl butamido triazone, Digalloyl trioleate, Diisopropyl methyl cinnamate, Ethyl dihydroxypropyl PABA, Ethylhexyl dimethoxy benzylidene dioxoimidazoline propionate, Ethylhexyl dimethyl PABA, Ethylhexyl triazone, Ferulic acid, Glyceryl ethylhexanoate dimethoxycinnamate, Glyceryl PABA, Isoamyl p-methoxycinnamate, Isopentyl trimethoxycinnamate trisiloxane, Isopropyl benzyl salicylate, Isopropyl methoxycinnamate, 4-Methylbenzylidene camphor, PABA, PEG-25 PABA, Pentyl dimethyl PABA, Phenyl benzimidazole sulfonic acid, Polyacrylamido methylbenzylidene camphor, Polysilicone-15, Salicylic acid, TEA salicylate and mixtures thereof. Preferably, UVB organic sunscreens that may be used in the leave-on composition are selected from OCR, MCX. phenyl benzimidazole sulphonic acid, ethyl hexyl salicylate and mixtures thereof.

When incorporated in the composition, UVB organic sunscreens may preferably be incorporated from 0.1 to 10 wt %, more preferably from 0.5 to 7 wt %, even more preferably from 1 to 5 wt %, further more preferably from 1 to 3.5 wt %, yet more preferably from 1 to 3 wt %, still more preferably 1 to 2.5 wt % in the leave-on composition.

Preferably, the leave-on composition may comprise a UVB/A sunscreen which are sunscreens that absorb both UVB and UVA radiations and prevent them from reaching the applied surface. Examples of UVB/A sunscreens that may be used in the composition include Benzophenone derivatives. Examples of such compounds are Benzophenone-1, Benzophenone-2, Benzophenone-3, Benzophenone-4, Benzophenone-5, Benzophenone-6, Benzophenone-8, Benzophenone-9, Beta-2-Glucopyranoxy propyl hydroxy benzophenone, Bis-ethylhexyloxyphenol methoxyphenyl triazine (Tinosorb S), Drometrizole, Drometrizole trisiloxane, Methylene bis-benzotriazolyl tetramethylbutylphenol (Tinosorb M).

Preferably, the leave-on composition comprises from 0.01 to 10 wt %, more preferably from 0.1 to 8 wt %, even more preferably from 1 to 6 wt %, further more preferably 1 to 5 wt %, still more preferably from 1 to 3 wt %, yet more preferably from 1 to 2.5 wt % of one or more UVB/A organic sunscreens.

Preferably, the composition may comprise an inorganic sunblock. Examples of inorganic sunblock that may be used in the composition include zinc oxide (ZnO), iron oxide, silica, such as fumed silica and titanium dioxide (TiO₂). Preferably, inorganic sunblock that may be used in the composition are selected from TiO₂, ZnO and mixtures thereof.

When incorporated the composition, inorganic sunblock may preferably be incorporated from 0.1 to 25% wt %, more preferably from 0.5 to 20 wt %, even more preferably from 1 to 15 wt %, further more preferably from 1 to 10 wt %, yet more preferably from 1 to 5 wt %, still more preferably 1 to 3 wt %, in the composition.

The leave-on composition may comprise one or more agents for providing glow to the skin. Examples of the agents include niacinamide, picolinamide, 4-alkyl substituted resorcinol, vitamin B6, vitamin C, vitamin A, glutathione precursors, galardin, adapalene, aloe extract, ammonium lactate, arbutin, azelaic acid, butyl hydroxy anisole, butyl hydroxy toluene, citrate esters, deoxyarbutin, 1,3-diphenyl propane derivatives, 2,5-dihydroxybenzoic acid and its derivatives, 2-(4-acetoxyphenyl)-1,3-dithiane, 2-(4-hydroxyphenyl)-1,3-dithiane, ellagic acid, gluco pyranosyl-1-ascorbate, gluconic acid, glycolic acid, green tea extract, 4-Hydroxy-5-methyl-3[2H]-furanone, 4-hydroxyanisole and its derivatives, 4-hydroxybenzoic acid derivatives, hydroxycaprylic acid, inositol ascorbate, lactic acid, lemon extract, linoleic acid, magnesium ascorbyl phosphate, 5-octanoyl salicylic acid, salicylic acid, 3,4,5-trihydroxybenzyl derivatives, acetylglucosamine, pitera extract, symwhite, calcium pantothenate (Melano-block), seppiwhite, soybean extract (bowman birk inhibitor), 12-hydroxystearic acid and mixtures thereof. When used in the composition, 12-hydroxystearic acid is used as a skin glow agent and not as a fatty acid.

Preferably, the agent for providing glow to the skin is selected from the group consisting of niacinamide, picolinamide, isonicotinamide, 12-hydroxystearic acid, 4-alkyl substituted resorcinol and mixtures thereof. Preferably the skin glow agent used in the leave-on composition is niacinamide. More preferably, the skin glow agent selected is 4-alkyl substituted resorcinol selected from the group consisting of 4-ethyl resorcinol (4-ER), 4-isopropyl resorcinol (4-IPR), 4-butyl resorcinol (4-BR), 4-pentyl resorcinol (4-PR), 4-phenylethyl resorcinol (4-PER), 4-HR and mixtures thereof. Even more preferably, the agent is selected from the group consisting of 4-ER, 4-HR and mixtures thereof. Further more preferably, the agent selected is 4-HR.

Preferably, the leave-on composition comprises from 0.001 to 10 wt %, more preferably from 0.01 to 8 wt %, even more preferably from 0.1 to 7 wt %, further more preferably from 0.1 to 5 wt %, still more preferably from 0.1 to 3 wt %, yet more preferably from 0.1 to 1 wt % and still further more preferably from 0.25 to 0.5 wt % of a skin glow agent.

The leave-on composition comprises a cosmetically acceptable vehicle. The cosmetically acceptable vehicle is preferably in the form of an oil, liquid, stick, cream, lotion, spray or gel. The cosmetically acceptable vehicle preferably includes ingredients like surfactant e.g. non-ionic surfactant, fatty acid, soap, water, polymer, emollients, solvents, powders, preservatives, or optional ingredients. Details on the various possible ingredients for inclusion in the cosmetically acceptable vehicle are given below.

Preferably, the leave-on composition comprises a nonionic surfactant. More preferably the nonionic surfactant is selected from those having HLB value in the range 9 to 20, preferably 10 to 19, more preferably 12 to 18, even more preferably 13 to 17 and yet more preferably 15 to 17.

HLB is calculated using the Griffin method wherein HLB=20×Mh/M wherein Mh is the molecular mass of the hydrophilic portion of the molecule and M is the molecular mass of the whole molecule, giving a result on an arbitrary scale of 0 to 20. Typical values for various surfactants are given below:
A value<10: Lipid soluble (water insoluble)
A value>10: Water soluble
A value from 4 to 8 indicates an anti-foaming agent
A value from 7 to 11 indicates a W/O (water in oil) emulsifier
A value from 12 to 16 indicates oil in water emulsifier
A value from 11 to 14 indicates a wetting agent
A value from 12 to 15 is typical of detergents
A value of 16 to 20 indicates a solubiliser or a hydrotrope.

Preferably, the nonionic surfactant having HLB value in the range 9 to 20 is selected from fatty alcohol ethoxylates, alkyl phenol ethoxylates, polyoxyethylene sorbitan alkyl esters and mixtures thereof. Preferably, the nonionic surfactants are ones with at least 9 alkylene oxide groups preferably at least 9 ethylene oxide groups.

Examples of fatty alcohol ethoxylates that may be used as nonionic surfactants in the composition include polyoxyethylene lauryl ether (HLB=16.9; commercially available as Brij® 35), polyoxyethylene (20) cetyl ether (HLB=16; commercially available as Brij® 58), polyethylene glycol octadecyl ether (HLB=18.8; commercially available as Brij® 700) and Laureth-9 (C12EO9; HLB=14.3; commercially available as Brij® L9).

Examples of alkyl phenol ethoxylates that may be used as nonionic surfactant in the composition include octylphenol ethoxylate (HLB=15.5; commercially available as Triton™ X165), octylphenol ethoxylate (HLB=17.6; commercially available as Triton™ X405) and octylphenol ethoxylate (HLB=18.4; commercially available as Triton™ X705).

Examples of polyoxyethylene sorbitan alkyl esters that may be used as the nonionic surfactant in the composition include polyoxyethylenesorbitan monolaurate (HLB=13.3; commercially available as Tween® 21), polyoxyethylenesorbitan monolaurate (HLB=16.7; commercially available as Tween® 20), Polyoxyethylenesorbitan monopalmitate (HLB=15.6; commercially available as Tween® 40) and polyoxyethylene sorbitan monostearate (HLB=14.9; commercially available as Tween® 60).

Even more preferably, the nonionic surfactant having HLB value in the range 9 to 20 that may be present in the composition is fatty alcohol ethoxylate with saturated carbon chain having HLB higher than 15.5.

Preferably, the leave-on composition comprises 0.5 to 5 wt %, more preferably 1 to 4 wt %, even more preferably from 2 to 3 wt % nonionic surfactant having HLB in the range 9 to 20.

Preferably, the leave-on composition comprises fatty acid. Fatty acids when present in a composition along with a soap provides the so-called vanishing cream effect, i.e. a composition, when applied on to the human skin, vanishes on the skin leaving behind no significant streaks of the composition. Preferably, the leave-on composition comprises fatty acids having 10 to 30, more preferably 12 to 25, even more preferably 14 to 20, further more preferably 16 to 18 carbon atoms. Examples of fatty acids that may be used in the composition include pelargonic, lauric, myristic, palmitic, stearic, isostearic, oleic, linoleic, arachidic, behenic, erucic acid and mixtures thereof. Preferably, the fatty acid that may be used is stearic acid or palmitic acid or a mixture thereof. The fatty acid in the present invention is preferably hystric acid which is substantially (generally about 90 to 95%) a mixture of stearic acid and palmitic acid in a ratio of between 55:45 to 45:55.

Preferably, the leave-on composition comprises from 2.25 to 25 wt %, more preferably from 4 to 22 wt %, even more preferably from 6 to 20 wt %, further more preferably from 8 to 19 wt % and still more preferably from 10 to 18 wt % and yet more preferably from 12 to 16 wt % fatty acid.

Preferably, the leave-on composition comprises soap. Soap, when present in combination with fatty acid in the composition, provides the vanishing effect described above. Preferably, soap in the leave-on composition is generally prepared by in-situ neutralization of fatty acid that may be present in the composition. Thus, it is preferred that the soap has a carbon chain length that corresponds to the chain length of fatty acid in the composition. The soap is formed from the fatty acid through use of alkali metal hydroxides e.g. sodium hydroxide or potassium hydroxide. Of the two, potassium hydroxide is more preferred. Thus, the soap is preferably a potassium soap (potassium salt of fatty acid).

Preferably, the leave-on composition comprises from 0.1 to 10 wt %, more preferably from 0.25% to 8 wt %, even more preferably from 0.5 to 7 wt %, further more preferably from 0.5 to 5 wt % soap, even further more preferably 0.5% to 3%.

Preferably, the leave-on composition comprises water in amount from 5 to 99.9 wt %, more preferably from 10 to 95 wt %, even more preferably from 15 to 90 wt %, further more preferably from 20 to 80 wt %, still more preferably 25 to 75 wt % and yet more preferably 30 to 70 wt %.

Preferably, the leave-on composition comprises a polymer. The polymer acts as thickener in the composition and improves sensorial properties of the composition.

The polymer is preferably selected from the following classes:
acrylate/R-methacrylate copolymer e.g. acrylates/steareth-20 methacrylate copolymer (commercially available as Aculyn™ 22) and acrylates/beheneth-25 methacrylate copolymer (commercially available as Aculyn™ 28),
acrylate/R-methacrylate crosspolymer e.g. acrylates/steareth-20 methacrylate crosspolymer (commercially available as Aculyn™ 88),
acrylates copolymer (commercially available as Aculyn™ 33),
acrylate/R-alkyl acrylate crosspolymer e.g. acrylates/C10-C30 alkyl acrylate crosspolymer (commercially available as Pemulen™ TR-2),
copolymer of ammonium acryloyldimethyltaurate with vinyl pyrrolidone (commercially available as Aristoflex® AVC),
copolymer of sodium acryloyldimethyltaurate with vinyl pyrrolidone (commercially available as Aristoflex® AVS); and
crosspolymer of acryloyldimethyltaurate with R-alkyl acrylate and methyacrylate e.g. Ammonium acryloyldimethyltaurate/beheneth-25 methacrylate crosspolymer (commercially available as Aristoflex® HMB and Aristoflex® BLV).

Preferably, the leave-on composition comprises 0.1 to 5 wt %, more preferably 0.25 to 4.5 wt %, even more preferably 0.5 to 4 wt %, further more preferably from 0.75 to 3.5 wt %, still more preferably from 0.75 to 2.75 wt % polymer.

Preferably, the leave-on composition comprises emollients. Examples of emollients that may be used in the leave-on composition include stearyl alcohol, glyceryl monoricinoleate, mink oil, cetyl alcohol, isopropyl isostearate, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, silicone oils such as dimethylpolysiloxane, din-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rape seed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isopropyl linoleate, lauryl lactate, myristyl lactate, decyl oleate, myristyl myristate and mixtures thereof.

Preferably, the leave-on composition comprises solvents. Examples of solvents that may be used in the composition include ethyl alcohol, isopropanol, acetone, ethylene glycol ono ethyl ether, diethylene glycol mono butyl ether, diethylene glycol mono ethyl ether and mixtures thereof.

Preferably, the leave-on composition comprises powders. Examples of powders that may be used in the composition include chalk, talc, fullers earth, kaolin, starch, gums, colloidal silica sodium polyacrylate, tetra alkyl and/or trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose, ethylene glycol monostearate and mixtures thereof.

Preferably, the leave-on composition comprises preservatives to protect against the growth of potentially harmful microorganisms. Examples of ingredients that may be used as preservatives in the composition include alkyl esters of para-hydroxybenzoic acid, hydantoin derivatives, propionate salts, and a variety of quaternary ammonium compounds. More preferably, ingredients that may be used as preservative in the composition are sodium benzoate, iodopropynyl butyl carbamate, methylisothiazolinone, iodopropynylbutylcarbamate, phenoxyethanol, methyl paraben, propyl paraben, imidazolidinyl urea, sodium dehydroacetate, ethylhexylglycerin, benzyl alcohol, alkane diols and mixtures thereof. The alkane diols that are suitable for use as preservative are C6-C12 alkanes that are vicinally substituted with hydroxy groups. Illustrative examples include 1,2-octane diol (caprylyl glycol), 2,3-octane diol, 1,2-nonane diol, 1,2-decane diol, 1,2-hexane diol, 3,4-octane diol, mixtures thereof or the like where caprylyl glycol is typically the most preferred.

When present in the leave-on composition, preservatives are added preferably in an amount 0.001 to 5 wt %, more preferably 0.01 to 3 wt % and most preferably 0.02 to 2 wt %, even most preferably 0.25 to 1.5%.

Preferably, the leave-on composition comprises a range of other optional ingredients that include antioxidants, binders, buffering agents, colorants, astringents, fragrance, opacifying agents, conditioners, exfoliating agents, pH adjusters, skin sensates, skin soothing agents, and skin healing agents.

The present invention relates to the method as described in detail herein above viz. the step of washing the surface with the wash-off composition followed by rinsing followed by applying a leave-on composition. It is preferable to dry the skin after the rinsing step by dabbing the surface with a towel to leave the surface substantially dry to touch. The step of applying the leave-on composition after the rinsing off step may be carried out anywhere from a minute to an hour after the rinsing step. Preferably the step of applying the leave-on composition is from one minute to 30 minutes, more preferably one minute to 10 minutes, further more preferably from one minute to five minutes after the rinsing step.

The invention also relates to a method of providing an SPF of 15, at least 20, preferably at least 30, more preferably at least 50, to the topical surface of the body. The invention also relates to a method of providing an UVAPF of 4 to skin, of at least 8, preferably at least 12, more preferably at least 16 to the topical surface of the body.

According to another aspect of the present invention, there is provided a kit comprising

(a) a wash-off composition comprising 0.1 to 10 wt % of a water soluble UVA sunscreen, 0.1 to 10 wt % of a water soluble UVB sunscreen, 3 to 80 wt %, preferably 6 to 80 wt % surfactant, and a cosmetically acceptable carrier, (b) a leave-on composition comprising a sunscreen and a cosmetically acceptable vehicle; and (c) Instructions for use.

The invention will now be demonstrated with the help of the following non-limiting examples.

### EXAMPLES

### Examples A-I. 1.2: Effect of the Method of the Invention (Examples 1,2) on the In-Vitro-SPF and UVA-PF as Compared to Methods Outside the Invention (Examples A-I)

The following skin cleansing compositions were prepared as shown in Table-1 below:

TABLE 1

| | Sample No: | | | | |
|---|---|---|---|---|---|
| | B | D | F | H | I |
| Ingredients | % | % | % | % | % |
| Sodium Laureth Sulfate | 12.86 | 3 | 3 | 3 | 12.86 |
| (Low C16) 70% | | | | | |
| Lauric Acid | 0 | 5.8 | 5.8 | 5.8 | 0 |
| Myristic Acid | 0 | 6.7 | 6.7 | 6.7 | 0 |
| Palmitic Acid | 0 | 2.1 | 2.1 | 2.1 | 0 |
| Hydroxypropyl Methyl | 0 | 0.2 | 0.2 | 0.2 | 0 |
| Cellulose | | | | | |
| Glycerin | 0.25 | 0.5 | 0.5 | 0.5 | 0.25 |
| Cocomonoethanol amide | 1.21 | 0 | 0 | 0 | 1.21 |
| 12 Hydroxy Stearic acid | 0 | 1.6 | 1.6 | 1.6 | 0 |
| Phenylbenzimidazole | 0 | 0 | 0 | 2 | 3 |
| Sulfonic Acid | | | | | |
| Disodium Phenyl | 0 | 0 | 0 | 0.75 | 1.2 |
| Dibenzimidazole | | | | | |
| Tetrasulfonate | | | | | |
| Ethylhexyl | 0 | 0 | 3 | 0 | 0 |
| Methoxycinnamate | | | | | |
| Avobenzone | 0 | 0 | 1.2 | 0 | 0 |
| Sodium Hydroxide 97% | 0 | 0 | 0 | 0 | 0.65 |
| Potassium Hydroxide | 0 | 4.1 | 4.1 | 4.76 | 0 |
| 85% | | | | | |
| Potassium Chloride | 0 | 3 | 3 | 3 | 0 |
| Sodium Chloride | 0.5 | 0 | 0 | 0 | 0.5 |
| Ethylene Glycol | 0 | 1.2 | 1.2 | 1.2 | 0 |
| Distearate | | | | | |
| Cocamidopropyl Betaine | 5.67 | 2.68 | 2.68 | 2.68 | 5.67 |
| (30%) | | | | | |
| Water | to 100 | to 100 | to 100 | to 100 | to 100 |

The following leave-on composition of the vanishing cream type was prepared as shown in Table-2 below:

TABLE 2

| | Sample No: |
|---|---|
| | A |
| Ingredients | Wt % |
| Glycerin | 1 |
| Potassium hydroxide (85%) | 0.57 |
| Fatty acid (Stearic & palmitic acid mix) | 17 |
| Cetyl alcohol | 0.53 |
| Dimethicone | 0.5 |
| Butyl methoxy dibenzoyl methane | 0.4 |
| Octyl methoxy cinnamate | 0.75 |
| isopropyl myristate | 0.75 |
| Titanium dioxide | 0.9 |
| DM water | To 100 |

Using the above samples, the following experiments were carried out

Example-A: The leave-on formulation of sample-A was applied on 6 micro meter roughed PMMA plates at 2 mg/cm² and allowed to dry for 20-30 minutes in dark. After drying, the PMMA plate was exposed to UV light and transmittance scan was recorded. This scan gives the transmittance as a function of wavelength (290-400 nm) for a given sample. For a single PMMA plate six different spots were scanned. The same was repeated for 3 PMMA plate samples. The data reported is thus an average over 18 readings. The reference transmittance scan was obtained using blank PMMA plate, with glycerine spread on it as control. The transmittance values were used to arrive at the SPF and UVA PF values using the software provided with the instrument.

For all the study SPF and UVAPF measurements carried out using Labsphere's UV-2000S Ultraviolet Transmittance Analyzer.

Examples B: A pre-wet vitro skin was applied with diluted wash-off product of Sample B. The vitro skin was rinsed off immediately post product application and kept for drying in dark. After the drying, vitro skin was exposed to UV light and transmittance scan was recorded. This scan gives the transmittance as a function of wavelength (290-400 nm) for a given sample. For a single vitro skin sample four different spots were scanned. The same was repeated for 3 vitro skin samples. The data reported is thus an average over 12 readings. The reference transmittance scan was obtained using blank vitro skin, with glycerine spread on it as control. The transmittance values were used to arrive at the SPF and UVA PF values using the software provided with the instrument.

Example-C: The experiment as per Example-B (Synthetic surfactant composition with no sunscreens) was first carried out. This was followed by applying a leave-on composition Sample-A using the following procedure. The vitro skin post washing with sample-B was applied with 2 mg/cm² of leave-on product Sample A on the same side of the vitro skin where cleansing was performed to mimic the consumer scenario. The vitro skin applied was then allowed to dry in the dark for 20-30 minutes to simulate recommended drying time from in vivo ISO 24444 methods. After drying, vitro skin was exposed to UV light and transmittance scan was recorded. This scan gives the transmittance as a function of wavelength (290-400 nm) for a given sample. For a single vitro skin sample four different spots were scanned. The same was repeated for 3 vitro skin samples. The data reported is thus an average over 12 readings. The reference transmittance scan was obtained using blank vitro skin, with glycerine spread on it as control. The transmittance values were used to arrive at the SPF and UVA PF values using the software provided with the instrument.

Example-D: Experiment as per Example B was carried out except that Sample D (Wash-off Composition comprising soap and synthetic surfactant with no sunscreens) was used instead of Sample B.

Example-E: Experiment as per Example C was carried out except that the vitro skin was first washed with Sample D followed by applying Sample A.

Example F: Experiment as per Example B was carried out except that Sample F (Wash-off Composition comprising soap and synthetic surfactant with oil soluble sunscreen) was used instead of Sample B.

Example G: Experiment as per Example C was carried out except that the vitro skin was first washed with Sample F followed by applying Sample A.

Example H: Experiment as per Example B was carried out except that Sample H (Wash-off Composition comprising soap and synthetic surfactant with water soluble sunscreen) was used instead of Sample B.

Example-1: Experiment as per Example C was carried out except that the vitro skin was first washed with Sample H followed by applying Sample A.

Example-I: Experiment as per Example B was carried out except that Sample I (Wash-off Composition comprising synthetic surfactant with water soluble sunscreen) was used instead of Sample B.

Example-2: Experiment as per Example C was carried out except that the vitro skin was first washed with Sample I followed by applying Sample A.

The SPF and UVAPF along with the respective standard deviations of the various is summarized in Table-3:

TABLE 3

| Example | In vitro SPF | STDV | UVA PF | STDV |
|---|---|---|---|---|
| Control | 0.99 | 0.01 | 0.0 | 0.0 |
| A | 13.81 | 1.07 | 2.7 | 0.8 |
| B | 0.94 | 0.01 | 0.0 | 0.0 |
| C | 10.77 | 2.60 | 2.2 | 1.0 |
| D | 0.93 | 0.01 | 0.0 | 0.0 |
| E | 8.26 | 2.07 | 1.6 | 1.5 |
| F | 0.99 | 0.01 | 0.0 | 0.0 |
| G | 7.80 | 1.06 | 1.3 | 0.6 |
| H | 6.38 | 0.61 | 1.2 | 0.8 |
| 1 | 40.85 | 5.13 | 6.5 | 3.7 |
| I | 16.82 | 1.74 | 3.5 | 2.0 |
| 2 | 89.69 | 14.36 | 15.7 | 7.1 |

The data in Table-3 above indicates that method as per the invention (Example-1 and 2) provides vastly superior SPF and UVAPF as compared to methods outside the invention (Examples A to I). Further, the data indicates that the use of a wash-off composition comprising only synthetic surfactant in the method of the invention (Example-2) is superior to use of a wash-off composition comprising both soap and synthetic surfactant in the method of the invention (Example-1).

Examples J-Q, 3,4: Effect of the method of the invention when the leave-on composition contains only UV-A (Sample J) sunscreen or only UV-B sunscreen (Sample-N):

Wash-off compositions as shown in the Table-4 below were first prepared:

TABLE 4

Sample

| | K | L |
|---|---|---|
| Ingredients | % | % |
| Acrylates copolymer (30%) | 6 | 6 |
| Sodium Laureth Sulfate (Low C16) 70% | 15 | 15 |
| Glycerin | 5 | 5 |
| Phenylbenzimidazole Sulfonic Acid | 0 | 3 |
| Disodium Phenyl Dibenzimidazole | 0 | 1.2 |
| Tetrasulfonate | | |
| Sodium Hydroxide 97% | 0.2 | 0.83 |
| Sodium Chloride | 0.16 | 0.16 |
| Cocamidopropyl Betaine 30% | 6 | 6 |
| Water | to 100 | to 100 |

Leave-on formulations as shown in Table-5 below were then prepared.

TABLE 5

Sample

| | J | N |
|---|---|---|
| Ingredients | Wt % | Wt % |
| Disodium EDTA | 0.1 | 0.1 |
| Glycerin | 1 | 1 |
| Potassium hydroxide (85%) | 0.57 | 0.57 |
| Fatty acid (Stearic & Palmitic | 17 | 17 |
| acid mix) | | |
| Cetyl alcohol | 0.53 | 0.53 |
| Dimethicone | 0.5 | 0.5 |
| Butyl methoxy dibenzoyl | 0 | 1.2 |
| methane | | |
| Octyl methoxy cinnamate | 2.25 | 0 |
| isopropyl myristate | 6 | 6 |
| Polyoxyethylene Lauryl | 2 | 2 |
| Ether | | |
| Acrylates Steareth-20 | 0.25 | 0.25 |
| Methacrylate Copolymer | | |
| Ammonium acryloyl dimethyl | 0.3 | 0.3 |
| taurate/beheneth 25 | | |
| methacrylate crosspolymer | | |
| DM water | to 100 | to 100 |

Example-J and N: The experiments using sample J (Leave-on composition containing a UVB sunscreen alone) and sample N (Leave-on composition containing a UVA sunscreen alone) were carried out in the same way and the measurements were made in the same way as has been described for Example-A except that Sample J and N were used instead of sample A.

Example-K: Experiment as per Example B was carried out except that Sample K (Wash-off Composition comprising synthetic surfactant with no sunscreens) was used instead of Sample B.

Example-L: Experiment as per Example B was carried out except that Sample L (Wash-off Composition comprising synthetic surfactant with water soluble sunscreens) was used instead of Sample B.

Example M: Experiment as per Example C was carried out except that the vitro skin was first washed with Sample K followed by applying Sample J.

Example 3: Experiment as per Example C was carried out except that the vitro skin was first washed with Sample L (Wash-off composition comprising synthetic surfactant and water soluble sunscreens) followed by applying Sample J.

Example-O: Experiment as per Example-K was repeated.

Example-P: Experiment as per Example-L was repeated.

Example Q: Experiment as per Example C was carried out except that the vitro skin was first washed with Sample K followed by applying Sample N.

Example 4: Experiment as per Example C was carried out except that the vitro skin was first washed with Sample L followed by applying Sample N.

The SPF and UVAPF along with the respective standard deviations of the various is summarized in Table-6:

TABLE 6

| Example | In vitro SPF | STDV | UVA PF | STDV |
|---|---|---|---|---|
| J | 9.83 | 0.15 | 1.34 | 0.01 |
| K | 1.03 | 0.03 | 1.05 | 0.03 |
| L | 16.34 | 2.10 | 2.30 | 0.18 |
| M | 8.59 | 1.39 | 1.23 | 0.02 |
| 3 | 32.44 | 1.99 | 3.11 | 0.20 |
| N | 4.05 | 0.07 | 13.85 | 0.45 |
| O | 0.97 | 0.01 | 1.02 | 0.01 |
| P | 17.90 | 2.91 | 2.42 | 0.28 |
| Q | 3.53 | 0.30 | 11.87 | 1.84 |
| 4 | 85.74 | 17.92 | 22.32 | 5.57 |

The data in Table-6 above indicates that method as per the invention (Example-3, and 4) provides vastly superior SPF and UVAPF as compared to methods outside the invention (Examples J to Q). Further the data in the above table indicates that even if only one type of sunscreen is included in the leave-on composition (either a UVA sunscreen as in Example-4 or a UVB sunscreen as in Example-3), synergistic benefit is obtained.

Examples-R to W and 5 to 7: Effect of the method of the invention when the leave-on composition contains three organic sunscreens (Sample R), four organic sunscreens (Sample S) or an inorganic sunblock (Sample T).

The following three samples of leave-on sunscreen compositions were prepared (Samples R to T) as shown in Tables 7-9 below:

TABLE 7
Leave-on vanishing cream composition
comprising three organic sunscreens:

| | Sample |
|---|---|
| | R |
| Ingredients | Wt % |
| Glycerin | 1.00 |
| Potassium hydroxide (85%) | 0.64 |
| Fatty acid (Stearic & palmitic acid mix) | 14.00 |
| Cetearyl Alcohol | 1.50 |
| Dimethicone | 1.00 |
| Butyl methoxy dibenzoyl methane | 1.25 |
| Ethylhexyl Salicylate | 2.0 |
| Octocrylene | 1.50 |
| Polyoxyethylene Lauryl Ether | 2.00 |
| Acrylates Copolymer | 0.50 |
| Acrylates Steareth-20 Methacrylate Copolymer | 0.40 |
| DM water | to 100 |

TABLE 8
Leave-on sunscreen composition in the form
of a gel containing four sunscreens:

| | Sample |
|---|---|
| | S |
| Ingredients | Wt % |
| Disodium EDTA | 0.05 |
| Humectant (Glycerin& Butylene Glycol) | 8.01 |
| Sodium Benzotriazolyl Butylphenol Sulfonate | 0.10 |
| Carbomer | 0.80 |
| Triethanolamine | 2.25 |
| Phenylbenzimidazole Sulfonic Acid | 1.50 |
| Ethylhexyl Salicylate | 2.00 |
| Octocrylene | 1.00 |
| Butyl Methoxydibenzoylmethane | 0.80 |
| Cyclohexasiloxane and cyclopentasiloxane | 7.00 |
| Mica & Colourant | 0.16 |
| Alcohol | 5.00 |
| Deionized Water | to 100 |

TABLE 9
Leave-on sunscreen composition comprising inorganic sunblock

| | Sample |
|---|---|
| | T |
| Ingredient | Wt % |
| Zinc Oxide | 22.40 |
| Caprylic/Capric Triglyceride | 10.92 |
| Glycerin | 3.00 |
| Caprylyl Caprylate/Caprate | 2.00 |
| Coco-Glucoside | 1.00 |
| Starch | 1.00 |
| Microcrystalline Cellulose | 0.85 |
| Phenoxyethanol | 0.60 |
| Arachidyl Glucoside | 0.52 |
| Polyglyceryl-3 Polyricinoleate | 0.32 |
| Glyceryl Oleate | 0.30 |
| Xanthan Gum | 0.20 |
| Cellulose Gum | 0.15 |
| Sodium Chloride | 0.10 |
| Water | to 100 |

The following experiments were carried out with the above leave-on samples (Samples R, S and T) along with wash-off composition samples L and K.

Example R: The experiment using sample R (Leave-on cream composition containing three sunscreens) was carried out in the same way and the measurements were made in the same way as has been described for Example-A except that Sample R was used instead of sample A.

Example-U: Experiment as per Example C was carried out except that the vitro skin was first washed with Sample K (Wash-off composition comprising synthetic surfactant and no sunscreens) followed by applying Sample R.

Example 5: Experiment as per Example C was carried out except that the vitro skin was first washed with Sample L (Wash-off composition comprising synthetic surfactant and water soluble sunscreens) followed by applying Sample R.

Example S: The experiment using sample S (Leave-on gel composition containing four sunscreens) was carried out in the same way and the measurements were made in the same way as has been described for Example-A except that Sample S was used instead of sample A.

Example-V: Experiment as per Example C was carried out except that the vitro skin was first washed with Sample K (Wash-off composition comprising synthetic surfactant and no sunscreens) followed by applying Sample S.

Example 6: Experiment as per Example C was carried out except that the vitro skin was first washed with Sample L (Wash-off composition comprising synthetic surfactant and water soluble sunscreens) followed by applying Sample S.

Example T: The experiment using sample T (Leave-on composition containing inorganic sunblock) was carried out in the same way and the measurements were made in the same way as has been described for Example-A except that Sample T was used instead of sample A.

Example-W: Experiment as per Example C was carried out except that the vitro skin was first washed with Sample K (Wash-off composition comprising synthetic surfactant and no sunscreens) followed by applying Sample T.

Example 7: Experiment as per Example C was carried out except that the vitro skin was first washed with Sample L (Wash-off composition comprising synthetic surfactant and water soluble sunscreens) followed by applying Sample T.

The SPF and UVAPF along with the respective standard deviations of the various is summarized in Table-10:

TABLE 10

| Example | SPF | STDV | UVA-PF-initial | STDV |
|---|---|---|---|---|
| R | 14.22 | 1.93 | 14.20 | 1.81 |
| U | 14.79 | 1.94 | 14.50 | 2.33 |
| 5 | 156.50 | 17.77 | 28.37 | 5.49 |
| S | 11.43 | 0.88 | 3.76 | 0.13 |
| V | 45.73 | 3.90 | 5.81 | 0.56 |
| 6 | 83.47 | 13.59 | 10.97 | 2.51 |
| T | 57.35 | 5.30 | 31.25 | 2.45 |
| W | 15.47 | 2.57 | 12.82 | 1.75 |
| 7 | 128.01 | 12.11 | 19.57 | 1.58 |

The data in Table-10 above indicates that the methods as per the invention (Examples 5-7) provide for vastly superior (and synergistic) improvements in both SPF and UVAPF over the respective individual steps of washing the skin with the wash off composition followed by applying a leave-on composition.

1. A method of providing high SPF to a topical surface of a body comprising:
(a) washing the surface with a wash-off composition comprising:
(i) 0.1 to 10 wt % of a water-soluble UVA sunscreen;
(ii) 0.1 to 10 wt % of a water-soluble UVB sunscreen;
(iii) 3 to 80 wt % of a surfactant; and
(iv) a cosmetically acceptable carrier;
(b) rinsing the surface with water; followed by
(c) applying a leave-on composition comprising a sunscreen and a cosmetically acceptable vehicle, on the surface,
wherein the solubility in water of the water-soluble UVA sunscreen and the water-soluble UVB sunscreen is higher than 10 g/l at 25° C.

2. The method as claimed in claim 1, wherein the water soluble UVB sunscreen is selected from phenyl benzimidazole sulphonic acid or benzylidene camphor sulfonic acid or mixtures thereof.

3. The method as claimed in claim 1, wherein the water soluble UVA sunscreen is selected from di sodium phenyl dibenzimidazole tetra sulfonate, terephthalylidene dicamphor sulfonic acid or mixtures thereof.

4. The method as claimed in claim 1, wherein the surfactant is a synthetic surfactant or a soap or mixtures thereof.

5. The method as claimed in claim 1, wherein the sunscreen in the leave-on composition is an organic UVA sunscreen, an organic UVB sunscreen, an inorganic sunblock or mixtures thereof.

6. The method as claimed in claim 5, wherein the organic UVA sunscreen is butyl methoxydibenzoylmethane or diethylamino hydroxybenzoyl hexyl benzoate; the organic UVB sunscreen is ethylhexyl methoxycinnamate, octocrylene, phenyl benzimidazole sulphonic acid, or ethyl hexyl salicylate; and the inorganic sunblock is zinc oxide or titanium dioxide.

7. The method as claimed in claim 1, wherein said cosmetically acceptable vehicle of the leave-on composition is in the form of a cream, stick, lotion, gel, or spray.

8. The method as claimed in claim 1, wherein the step of applying the leave-on composition is carried out one minute to an hour after the step of rinsing said surface with water.

9. The method as claimed in claim 1 for obtaining an SPF of at least 15.

10. The method as claimed in claim 1 for obtaining UVAPF of at least 4.

11. A kit comprising:
(a) a wash-off composition comprising 0.1 to 10 wt % of a water soluble UVA sunscreen, 0.1 to 10 wt % of a water soluble UVB sunscreen, 3 to 80 wt % of a surfactant, and a cosmetically acceptable carrier,
(b) a leave-on composition comprising a sunscreen and a cosmetically acceptable vehicle; and
(c) instructions for use.

12. The method as claimed in claim 1, wherein the surface is dried after the rinsing step.

13. The method as claimed in claim 1, wherein the leave-on composition is applied from a minute to an hour after the rinsing step.

14. The method as claimed in claim 1, wherein the wash-off composition comprises 6 to 80 wt % of a surfactant.

15. The method as claimed in claim 1, wherein the surfactant comprises an anionic surfactant.

16. The method as claimed in claim 1, wherein the surfactant comprises an amphoteric surfactant.

17. The method as claimed in claim 1, wherein the wash-off composition further comprises an adjuvant system.

18. The method as claimed in claim 1, wherein the wash-off composition further comprises polymers.

19. The method as claimed in claim 1, wherein the leave-on composition further comprises at least one additional agent selected from niacinamide, picolinamide, 4-alkyl substituted resorcinol, vitamin B6, vitamin C, vitamin A, glutathione precursors, galardin, adapalene, aloe extract, ammonium lactate, arbutin, azelaic acid, butyl hydroxy anisole, butyl hydroxy toluene, citrate esters, deoxyarbutin, 1,3-diphenyl propane derivatives, 2,5-dihydroxybenzoic acid and its derivatives, 2-(4-acetoxyphenyl)-1,3-dithiane, 2-(4-hydroxyphenyl)-1,3-dithiane, ellagic acid, gluco pyranosyl-1-ascorbate, gluconic acid, glycolic acid, green tea extract, 4-Hydroxy-5-methyl-3[2H]-furanone, 4-hydroxyanisole and its derivatives, 4-hydroxybenzoic acid derivatives, hydroxycaprylic acid, inositol ascorbate, lactic acid, lemon extract, linoleic acid, magnesium ascorbyl phosphate, 5-octanoyl salicylic acid, salicylic acid, 3,4,5-trihydroxybenzyl derivatives, acetylglucosamine, pitera extract, symwhite, calcium pantothenate, seppiwhite, soybean extract, 12-hydroxystearic acid and mixtures thereof.
